(19) United States
(12) Patent Application Publication
(10) Pub. No.: US 2013/0184532 A1
(43) Pub. Date: Jul. 18, 2013

(54) ENDOSCOPIC METHOD FOR VIEWING A SINUS OPENING

(71) Applicants: Eric A. Goldfarb, Belmont, CA (US); Thomas R. Jenkins, Alameda, CA (US); John Y. Chang, Los Altos, CA (US); Joshua Makower, Los Altos, CA (US)

(72) Inventors: Eric A. Goldfarb, Belmont, CA (US); Thomas R. Jenkins, Alameda, CA (US); John Y. Chang, Los Altos, CA (US); Joshua Makower, Los Altos, CA (US)

(21) Appl. No.: 13/551,898

(22) Filed: Jul. 18, 2012

Related U.S. Application Data
(60) Provisional application No. 61/511,304, filed on Jul. 25, 2011.

(51) Int. Cl. A61B 1/00

(52) U.S. Cl. CPC A61B 1/00183
USPC 600/171; 600/160

(57) ABSTRACT

The invention is an endoscopic method for viewing a target portion of a patient's anatomy with access through the oral or nasal cavity. The method includes adjusting a variable direction of view endoscope to a direction of view of between about 0 degrees and 20 degrees relative to a longitudinal axis of the endoscope and a shaft orientation pointing toward the target portion of the patient's anatomy. The endoscope is then introduced straight into the oral or nasal cavity of a patient until the endoscope is positioned in the back of the cavity. The direction of view is adjusted to view the target portion of the patient's anatomy.

### FIELD OF THE INVENTION

The present invention relates to achieving visualization of desired locations within the ear, nose, throat, and paranasal sinuses.

### BACKGROUND OF THE INVENTION

Functional endoscopic sinus surgery (FESS) is currently the most common type of surgery used to treat chronic sinusitis. In a typical FESS procedure, an endoscope is inserted into the nostril along with one or more surgical instruments. The surgical instruments are then used to cut tissue and/or bone, cauterize, suction, etc. In most FESS procedures, the natural ostium (e.g. opening) of at least one paranasal sinus is surgically enlarged to improve drainage from the sinus cavity. The endoscope provides a direct line-of-sight view whereby the surgeon is typically able to visualize some but not all anatomical structures within the surgical field. Under visualization through the endoscope, the surgeon may remove diseased or hypertrophic tissue or bone and may enlarge the ostia of the sinuses to restore normal drainage of the sinuses. FESS procedures can be effective in the treatment of sinusitis and for the removal of tumors, polyps and other aberrant growths from the nose.

In order to adequately view the operative field through any endoscope and/or to allow insertion and use of rigid instruments, many FESS procedures of the prior art have included the surgical removal or modification of normal anatomical structures. For example, in many prior art FESS procedures, a total uncinectomy (e.g., removal of the uncinate process) is performed at the beginning of the procedure to allow visualization and access of the maxillary sinus ostium and/or ethmoid bulla and to permit the subsequent insertion of the rigid surgical instruments. Indeed, in most traditional FESS procedures, if the uncinate process is allowed to remain, such can interfere with endoscopic visualization of the maxillary sinus ostium and ethmoid bulla, as well as subsequent dissection of deep structures using the available rigid instrumentations.

A device that provides an alternative to the surgical removal or modification of anatomical structures to adequate view the operative field through an endoscope is described in US Patent Publication No. 2010/0030031 entitled Swing Prism Endoscope, which is hereby incorporated herein in its entirety. The endoscope described therein allows the user to view anatomy, such as a paranasal sinus ostium, without using/exchanging multiple endoscopes during a procedure or removing tissue as may be required in a traditional FESS procedure. Such a scope may also allow a physician to view anatomy and surgical tools without fluoroscope of image guidance systems, or at least with limited use of such systems so that a procedure might be performed in a clinic or procedure room setting rather than in an operating room. Procedures must be developed, however, to optimize the use of this and other such devices for access and visualization of particular tortuous anatomy in intracranial procedures such as ear, nose and throat procedures like paranasal sinus surgery.

### SUMMARY OF THE INVENTION

Accordingly, we have developed methods for using endoscopes such as the Swing Prism Endoscope described in US Patent Publication No. 2010/0030031 to view tortuous anatomy, including but not limited to the maxillary sinus ostium, the frontal sinus ostium or a frontal sinus outflow tract, a sphenoid sinus ostium, or a natural or man-made opening of an ethmoid sinus.

In one aspect, the invention is an endoscopic method for viewing a right maxillary sinus ostia. The method includes adjusting a variable direction of view endoscope to a first direction of view of between about 0 degrees and 20 degrees relative to a longitudinal axis of the endoscope and a shaft orientation to between about 60 degrees and 120 degrees relative to the longitudinal axis of the endoscope, introducing the variable direction of view endoscope straight into a right nasal cavity of a patient until the endoscope is positioned in the back of the right nasal cavity, and adjusting the direction of view to a second direction of view of about 60 degrees to 100 degrees relative to the longitudinal axis to view the right maxillary sinus ostia within the right nasal cavity. The right maxillary sinus ostia may be visible behind an uncinate with or without minor adjustment of the direction of view or shaft orientation of the endoscope.

In one embodiment, the first direction of view in the method for viewing the right maxillary sinus ostia is adjusted to between about 5 and 15 degrees relative to the longitudinal axis of the endoscope.

In a further embodiment, the second direction of view in the method for viewing the right maxillary sinus ostia is adjusted to between about 80 and 90 degrees relative to the longitudinal axis of the endoscope.

In another embodiment, the shaft orientation in the method for viewing the right maxillary sinus ostia is adjusted to between about 80 and 100 degrees relative to the longitudinal axis of the endoscope.

In another aspect, the invention is an endoscopic method for viewing a left maxillary sinus ostia. The method includes adjusting a variable direction of view endoscope to a first direction of view of between about 0 degrees and 20 degrees relative to a longitudinal axis of the endoscope and a shaft orientation to between about −60 degrees and −120 degrees relative to the longitudinal axis of the endoscope, introducing the variable direction of view endoscope straight into a left nasal cavity of a patient until the endoscope is positioned in the back of the left nasal cavity, and adjusting the direction of view to a second direction of view of about 60 degrees to 100 degrees relative to the longitudinal axis to view the left maxillary ostia within the left nasal cavity. The left maxillary sinus ostia may be visible behind an uncinate with or without minor adjustment of the direction of view or shaft orientation of the endoscope.

In one embodiment, the first direction of view for viewing the left maxillary sinus ostia is adjusted to between about 5 and 15 degrees relative to the longitudinal axis of the endoscope.

In a further embodiment, the second direction of view for viewing the left maxillary sinus ostia is adjusted to between about 80 and 90 degrees relative to the longitudinal axis of the endoscope.

In another embodiment, the shaft orientation for viewing the left maxillary sinus osita is adjusted to between about −80 and −100 degrees relative to the longitudinal axis of the endoscope.

In still another aspect, the invention is an endoscopic method for viewing a sphenoid sinus ostia. The method includes adjusting a variable direction of view endoscope to a direction of view of between about 0 degrees and 20 degrees relative to a longitudinal axis of the endoscope, and introducing the variable direction of view endoscope straight into a nasal cavity of a patient until the endoscope is positioned in the back of the nasal cavity. The sphenoid sinus ostia may be visible with or without minor adjustment of the direction of view or shaft orientation of the endoscope

In one embodiment, the direction of view for viewing the sphenoid sinus ostia is adjusted to between about 5 and 15 degrees relative to the longitudinal axis of the endoscope.

In another aspect, the method for viewing a sphenoid sinus ostia includes adjusting a variable direction of view endoscope to a direction of view of between about 20 degrees and 70 degrees relative to a longitudinal axis of the endoscope, and introducing the variable direction of view endoscope straight into a nasal cavity of a patient until the endoscope is positioned below a middle turbinate of the nasal cavity. The sphenoid sinus ostia may be visible with or without minor adjustment of the direction of view or shaft orientation of the endoscope.

In one embodiment, the direction of view for viewing the sphenoid sinus ostia is adjusted to between about 30 and 40 degrees relative to the longitudinal axis of the endoscope.

In yet another aspect, the invention is an endoscopic method for viewing a right frontal sinus ostia. The method comprises adjusting a variable direction of view endoscope to a first direction of view of between about 0 degrees and 20 degrees relative to a longitudinal axis of the endoscope and a shaft orientation to between about −20 degrees and 20 degrees relative to the longitudinal axis of the endoscope, introducing the variable direction of view endoscope straight into a right nasal cavity of a patient until the endoscope is positioned in the back of the right nasal cavity, and adjusting the direction of view to a second direction of view of about 30 degrees to 90 degrees relative to the longitudinal axis to view the right fronal sinus ostia within the right nasal cavity. The right frontal sinus ostia may be visible with or without minor adjustment of the direction of view or shaft orientation of the endoscope.

In one embodiment, the first direction of view for viewing the right frontal sinus ostia is adjusted to between about 5 and 15 degrees relative to the longitudinal axis of the endoscope.

In a further embodiment, the second direction of view for viewing the right frontal sinus ostia is adjusted to between about 45 and 90 degrees relative to the longitudinal axis of the endoscope.

In another embodiment, the shaft orientation for viewing the right frontal sinus ostia is adjusted to between about −10 and 10 degrees relative to the longitudinal axis of the endoscope.

In another aspect, the invention is an endoscopic method for viewing a left frontal sinus ostia. The method includes adjusting a variable direction of view endoscope to a first direction of view of between about 0 degrees and 20 degrees relative to a longitudinal axis of the endoscope and a shaft orientation to between about −20 degrees and 20 degrees relative to the longitudinal axis of the endoscope, introducing the variable direction of view endoscope straight into a left nasal cavity of a patient until the endoscope is positioned in the back of the left nasal cavity, and adjusting the direction of view to a second direction of view of about 30 degrees to 90 degrees relative to the longitudinal axisto view the left fronal sinus ostia within the left nasal cavity. The left frontal sinus ostia may be visible with or without minor adjustment of the direction of view or shaft orientation of the endoscope.

In one embodiment, the first direction of view for viewing the left frontal sinus ostia is adjusted to between about 5 and 15 degrees relative to the longitudinal axis of the endoscope.

In a further embodiment, the second direction of view for viewing the left frontal sinus ostia is adjusted to between about 45 and 90 degrees relative to the longitudinal axis of the endoscope.

In another embodiment, the shaft orientation for viewing the left frontal sinus ostia is adjusted to between about −10 and 10 degrees relative to the longitudinal axis of the endoscope.

In a further aspect, the invention is an endoscopic method for viewing a target portion of a patient's anatomy with access through a nasal cavity. The method includes adjusting a variable direction of view endoscope to a direction of view of between about 0 degrees and 20 degrees relative to a longitudinal axis of the endoscope and a shaft orientation pointing toward the target portion of the patient's anatomy, introducing the variable direction of view endoscope straight into the nasal cavity of a patient until the endoscope is positioned in the back of the nasal cavity, and adjusting the direction of view and optionally the shaft orientation to view the target portion of the patient's anatomy.

In still another aspect, the invention is an endoscopic method for viewing a target portion of a patient's anatomy with access through the oral cavity. The method includes adjusting a variable direction of view endoscope to a direction of view of between about 0 degrees and 20 degrees relative to a longitudinal axis of the endoscope and a shaft orientation pointing toward the target portion of the patient's anatomy, introducing the variable direction of view endoscope straight into the oral cavity of a patient until the endoscope is positioned in the back of the oral cavity, and adjusting the direction of view to and optionally the shaft orientation view the target portion of the patient's anatomy.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings, in which like numerals indicate like elements, of which:

FIG. 1 is a perspective view of a swing prism endoscope according to one embodiment of the present invention;

FIG. 2 is a top view of a proximal body member or handle of a swing prism endoscope equipped with turning dials to control the rotation of the endoscope shaft and rotation of the swing prism;

FIGS. 3A-3B are schematic views of the view from side (FIG. 3A) and top (FIG. 3B) using a swing prism endoscope with the elongate shaft direction and direction of view shown;

FIGS. 4A-4B are schematic views of the view from side (FIG. 4A) and top (FIG. 4B) using a swing prism endoscope with the elongate shaft direction and direction of view shown;

FIGS. 5A-5B are schematic views of the view from side (FIG. 5A) and top (FIG. 5B) using a swing prism endoscope with the elongate shaft direction and direction of view shown;

FIGS. 6A-6B are schematic views of the view from side (FIG. 6A) and top (FIG. 6B) using a swing prism endoscope with the elongate shaft direction and direction of view shown;

FIGS. 7A-7B are schematic views of the view from side (FIG. 7A) and top (FIG. 7B) using a swing prism endoscope with the elongate shaft direction and direction of view shown;

### DETAILED DESCRIPTION OF INVENTION

In the following description, where a range of values is provided, each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are now described. All publications mentioned herein are incorporated herein by reference to disclose and described the methods and/or materials in connection with which the publications are cited.

As used herein and in the appended claims, the singular forms “a”, “an”, and “the” include plural referents unless the context clearly dictates otherwise. Thus, for example, reference to “a channel” includes a plurality of such channels and reference to “the endoscope” includes reference to one or more endoscopes and equivalents thereof, and so forth.

The publications discussed herein are provided solely for their disclosure prior to the filing date of the present application. Nothing herein is to be construed as an admission that the present disclosure is not entitled to antedate such publication by virtue of prior invention. Further, the dates of publication provided may be different from the actual publication dates which may need to be independently confirmed.

The following detailed description, the accompanying drawings and the above-set-forth Brief Description of the Drawings are intended to describe some, but not necessarily all, examples of embodiments of the disclosure. The contents of this detailed description do not limit the scope of the disclosure in any way.

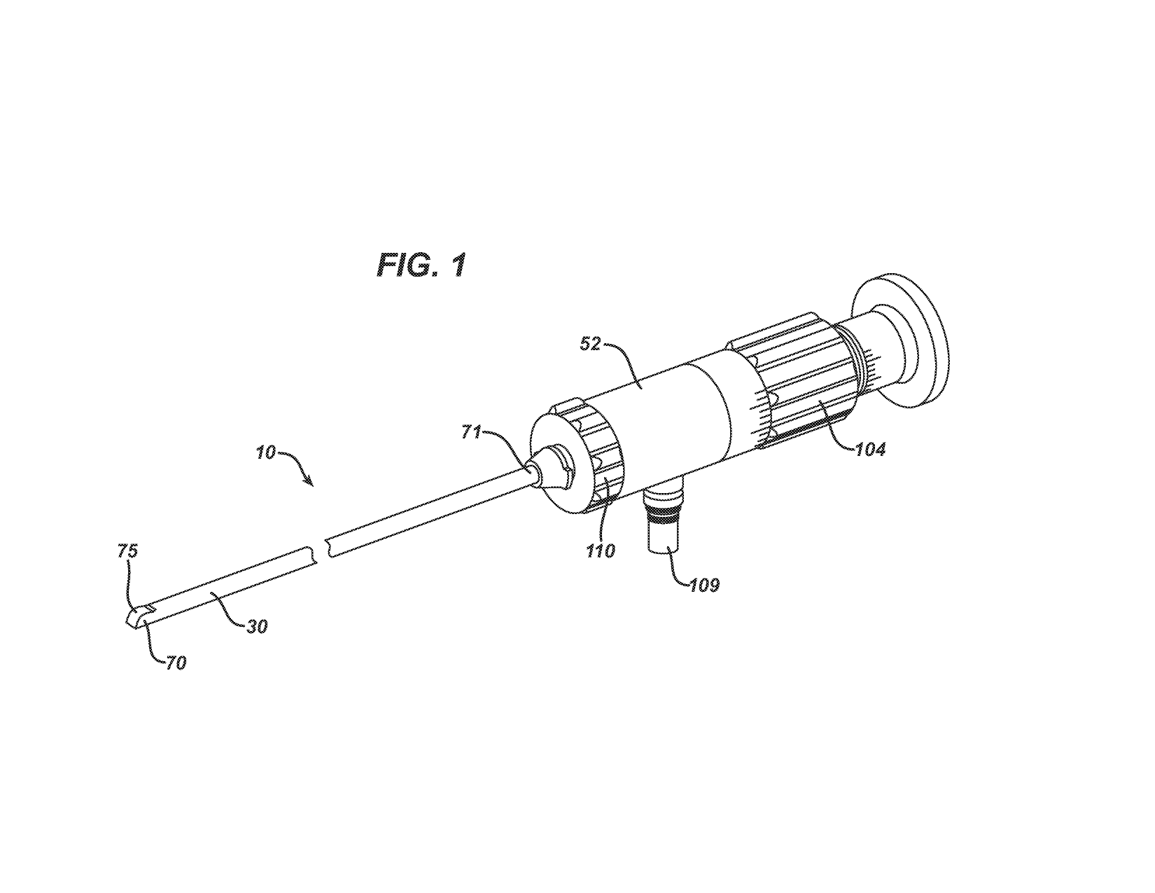

FIG. 1 shows a variable degree of view endoscope 10 according to one embodiment. The endoscope 10 may include an elongate shaft 30 with a distal end 70 and a proximal end 71, the latter being attached to a proximal body member or handle 52 that can be adapted to engage and attach to the adjustable scope/lock extension, and a swing prism for adjusting the viewing angle of the endoscope. Such swing prism is described in US Patent Publication No. 2010/0030031.

In the embodiment shown in FIG. 1, shaft dial 110 is disposed on the handle 52 of the endoscope 10, and when rotated, functions to control rotation of the endoscope shaft 30. All descriptions herein will be made with the understanding that the light post 109 is facing down. If the light post 109 is pointed in any other direction, the shaft 30 will be rotated to accommodate the change in direction. As shown in FIG. 2, a shaft dial indicator 112 on the shaft dial 110 is shown to indicate the relative position of the endoscope shaft 30. More particularly, the indicator 112 on the shaft dial 110 indicates the relative position of the window 75 at the distal portion 70 of the endoscope shaft. As the dial 110 is rotated to the right, the shaft 30 rotates to the right and as it is rotated to the left, the shaft 30 rotates accordingly. The shaft alignment marker 114 is located on the handle 52 near the shaft dial 110 and when aligned with the shaft dial indicator, indicates that the shaft is in the minimum or up position. As further shown in the embodiment of FIG. 1, the direction of view dial 104 is disposed on the handle of the endoscope 10, and when rotated, functions to control movement of the prism (not shown) and thus the direction of view of the endoscope. As shown in FIG. 2, a direction of view dial marker 108 on the direction of view dial 104 is shown to indicate the relative angle of the prism. The direction of view scale 107 on the handle 52 adjacent to the direction of view dial 104 indicate relative angle of the swing prism anywhere from approximately 0 to 10 degrees (when the direction of view dial indicator 108 is aligned with the minus sign on the direction of view scale 107 and has a hard stop) to approximately 90 to 100 degrees (when the direction of view dial indicator 108 is aligned with the plus sign on the direction of view scale 107 and has a hard stop).

In some embodiments, the endoscope may have a range of directions of view from about 0 degrees to about 100 degrees and more likely from about 10° to about 90°. The tolerance of −10 and 8 degrees on the low end is from about 0 degrees to about 18 degrees and at the high end is from about 82 degrees to about 100 degrees. In some embodiments, the endoscope shaft may be rotated a total of about 320 to 340 degrees, that is up to plus 160 to 170 degrees to the right of center (clockwise to the plus sign) and up to minus 160 to 170 degrees to the left of center (counter-clockwise to the minus sign). The shaft cannot be rotated past the plus and minus signs as there is a hard stop at those positions. In various embodiments, the endoscope 10 may have any of a number of different combinations and ranges of directions of view, fields of view and total ranges of view and inciators that specify the directions of view, fields of view and total ranges of view.

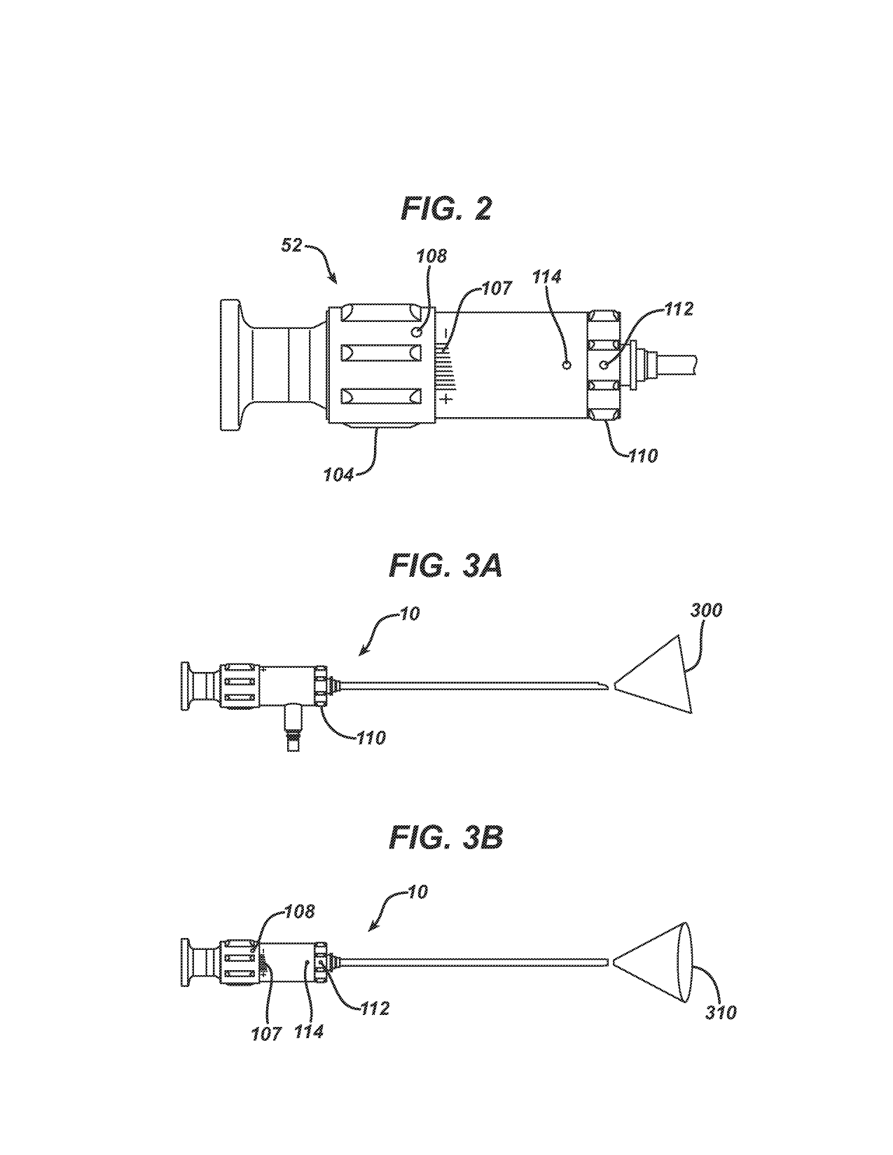

As shown in FIGS. 3A and 3B, in one embodiment, in order to initially position the swing prism endoscope 10 with the oral or nasal anatomy, the direction of view dial 104 is at the minimum position. In this way, the medical professional user is looking forward when entering the anatomy. The shaft dial 110 is initially positioned in the direction of the target anatomy so that the user may not have to manipulate the shaft dial 110 when the endoscope 10 is inside the patient, although such manipulation is easily accomplished. FIG. 3A is a side view of the endoscope showing the minimum (approximately 10 degrees) direction of view and the nominal shaft position (approximately 0 degrees). FIG. 3B shows the alignment of the direction of view dial indicator 108 with the minimum point (the minus sign) on the direction of view scale 107 and the alignment of the shaft alignment indicator 112 and the shaft alignment marker 114. Cones 300 and 310 show the view orientation with the shaft alignment and prism orientation as described from the side (300) and from the top (310).

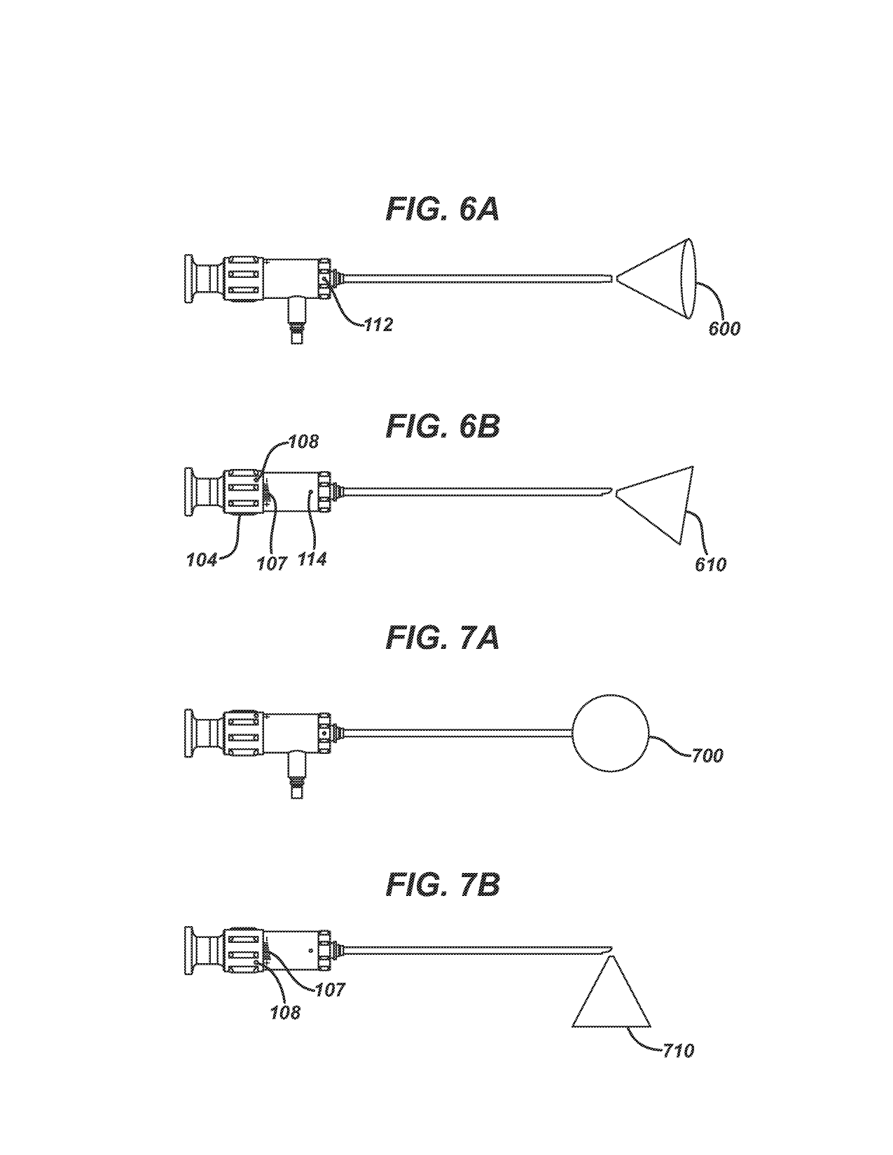

To view the left or right sphenoid sinus, the method is as follows. Initially, align the swing prism endoscope 10 with the direction of view dial 104 at the minimum position (approximately 10 degrees, or between 0 and 20 degrees, or between about 5 and 15 degrees). In this way, the medical professional user is looking forward when entering the anatomy. The shaft dial 110 is initially positioned in the direction of the target anatomy so that the user will not have to manipulate the shaft dial 110 when the endoscope 10 is inside the patient, but in this case, since the target anatomy is straight, the shaft 30 can be in any position for viewing the sphenoid sinus ostia. FIG. 3A is a side view of the endoscope showing the minimum (approximately 10 degrees) direction of view and the nominal shaft position (approximately 0 degrees, or between about −10 and 10 degrees or between about −20 and 20 degrees). FIG. 3B shows the alignment of the direction of view dial indicator 108 with the minimum point (the minus sign) on the direction of view scale 107 and the alignment of the shaft alignment indicator 112 and the shaft alignment marker 114. Cones 300 and 310 show the view orientation with the shaft alignment and prism orientation as described from the side (300) and from the top (310). Alternatively, in order to view the sphenoid sinus, the shaft can be positioned at the side position (for the right sphenoid sinus at approximately 90 degrees, or between about 80 and 100 degrees or between about 60 and 120 degrees and for the left sphenoid sinus at approximately −90 degrees, or between about −80 and −100 degrees or between about −60 and −120 degrees) as shown in FIGS. 6A and 6B with view orientations indicated at cones 600 and 610 or any other position since the medical professional user will be looking forward so long as the direction of view dial 104 is in the minimum position. Following adjustment of the shaft dial 110 and the direction of view dial 104 to view either the right or left sphenoid sinus ostia, the method includes introducing the variable direction of view endoscope straight into a nasal cavity of the patient until the endoscope is positioned in the back of the nasal cavity. The sphenoid sinus ostia may be visible with or without minor adjustment of the direction of view or shaft orientation of the endoscope. Where it is difficult to view the sphenoid sinus, the endoscope may be positioned below the middle turbinate and the direction of view adjusted to between about 20 and 70 degrees, or between about 30 and 40 degrees or about 30 degrees.

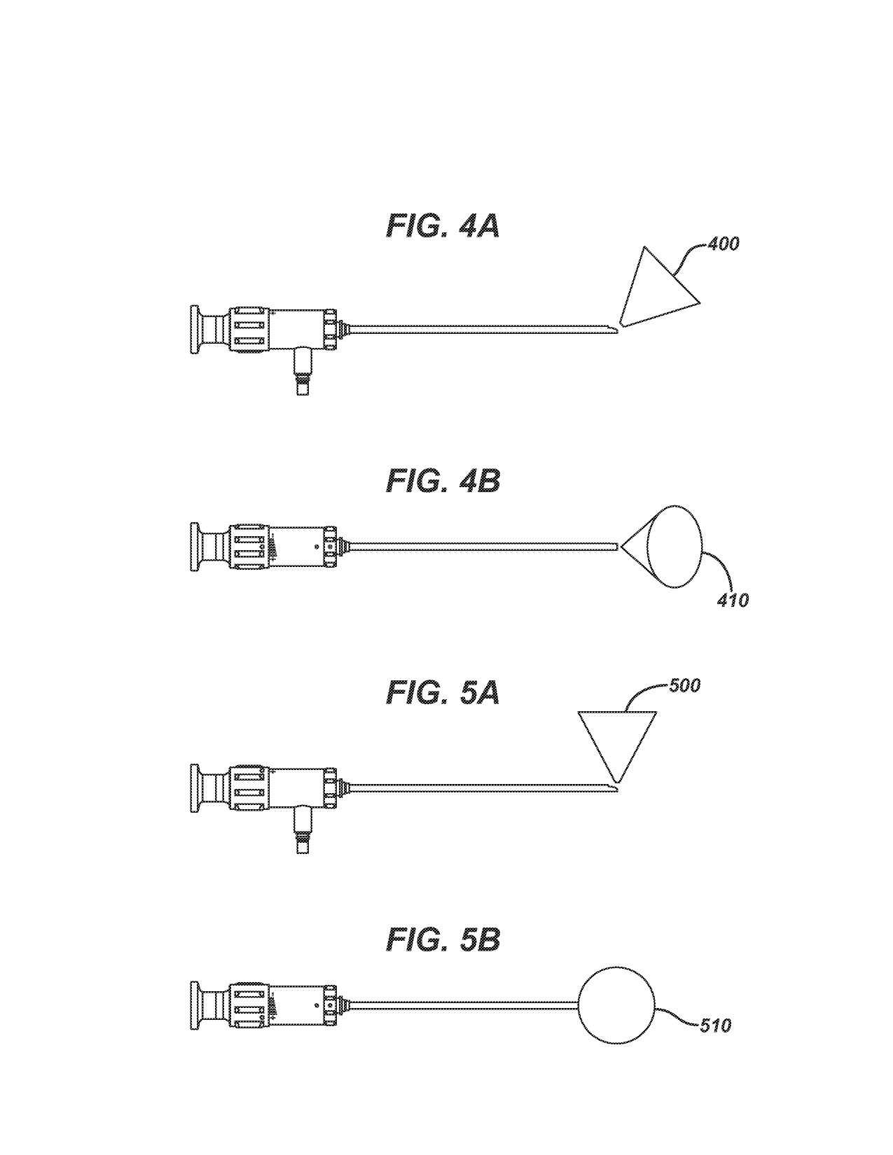

To view the right frontal sinus, the method is as follows. Initially, align the swing prism endoscope 10 with the direction of view dial 104 at the minimum position. In this way, the medical professional user is looking forward when entering the anatomy. FIG. 3A is a side view of the endoscope showing the minimum (approximately 10 degrees) direction of view and the nominal shaft position (approximately 0 degrees). FIG. 3B shows the alignment of the direction of view dial indicator 108 with the minimum point (the minus sign) on the direction of view scale 107 and the alignment of the shaft alignment indicator 112 and the shaft alignment marker 114. Cones 300 and 310 show the view orientation with the shaft alignment and prism orientation as described from the side (300) and from the top (310). After aligning the shaft dial indicator 112 with the shaft alignment marker 114 and the direction of view dial indicator 108 to the minus on the direction of view scale 107, introduce the variable direction of view endoscope 10 straight into the right nostril of the patient until the endoscope is positioned in the back of the nasal cavity. Once the tip is positioned in the back of the nasal cavity, the direction of view dial is positioned to approximately 45 degrees or between about 30 degrees and 90 degrees or between about 45 degrees and about 90 degrees, i.e. between the minimum (minus) and maximum (plus) positions (See FIGS. 4A and 4B showing view cones 400 and 410), and the maximum (plus) position (see FIGS. 5A and 5B showing view cones 500 and 510) to view the right frontal recess. The right frontal sinus ostia may be visible with or without minor adjustment of the direction of view or shaft orientation of the endoscope.

To view the left frontal sinus, the method is as follows. Initially, align the swing prism endoscope 10 with the direction of view dial 104 at the minimum position. In this way, the medical professional user is looking forward when entering the anatomy. FIG. 3A is a side view of the endoscope showing the minimum (approximately 10 degrees) direction of view and the nominal shaft position (approximately 0 degrees). FIG. 3B shows the alignment of the direction of view dial indicator 108 with the minimum point (the minus sign) on the direction of view scale 107 and the alignment of the shaft alignment indicator 112 and the shaft alignment marker 114. Cones 300 and 310 show the view orientation with the shaft alignment and prism orientation as described from the side (300) and from the top (310). After aligning the shaft dial indicator 112 with the shaft alignment marker 114 and the direction of view dial indicator 108 to the minus on the direction of view scale 107, introduce the variable direction of view endoscope 10 straight into the left nostril of the patient until the endoscope is positioned in the back of the nasal cavity. Once the tip is positioned in the back of the nasal cavity, the direction of view dial is positioned to approximately 45 degrees or between about 30 and 90 degrees or between about 45 and 90 degrees (See FIGS. 4A and 4B showing view cones 400 and 410) and the maximum position (see FIGS. 5A and 5B showing view cones 500 and 510) to view the left frontal recess. The left frontal sinus ostia may be visible with or without minor adjustment of the direction of view or shaft orientation of the endoscope.

To view the right maxillary sinus, the method is as follows. Initially, align the swing prism endoscope 10 with the direction of view dial 104 at the minimum position (approximately 10 degrees) and the shaft dial 110 to approximately 90 degrees or between about 60 and 120 degrees or between about 80 and 100 degrees so that the shaft dial indicator 112 is 90 degrees from the shaft alignment mark 114. The shaft 30 will be at the side shaft position (approximately 90 degrees). FIGS. 6A and 6B show the alignment of the direction of view dial indicator 108 with the minimum point (the minus sign) on the direction of view scale 107 and the alignment of the shaft alignment indicator 112 relative to the shaft alignment marker 114. Cones 600 and 610 show the view orientation with the shaft alignment and prism orientation as described from the side (600) and from the top (610). After aligning the shaft dial indicator 112 relative to the shaft alignment marker 114 and the direction of view dial indicator 108 to the minus on the direction of view scale 107, introduce the variable direction of view endoscope 10 straight into the right nostril of the patient until the endoscope is positioned in the back of the nasal cavity. Once the tip is positioned in the back of the nasal cavity, the direction of view dial is positioned to 90 degrees by aligning the view dial indicator 108 with the maximum point (the plus sign) on the direction of view scale 107 (see FIGS. 7A and 7B showing view cones 700 and 710) to view the behind the uncinate and into the right maxillary sinus ostia. The right maxillary sinus ostia may be visible with or without minor adjustment of the direction of view or shaft orientation of the endoscope.

To view the left maxillary sinus, the method is as follows. Initially, align the swing prism endoscope 10 with the direction of view dial 104 at the minimum position (approximately 10 degrees) and the shaft dial 110 to approximately −90 degrees or between about −60 and −120 degrees or between about −80 and −100 degrees so that the shaft dial indicator 112 is approximately 90 degrees from the shaft alignment mark 114. The shaft 30 will be at the side shaft position (approximately 90 degrees). After aligning the shaft dial indicator 112 relative to the shaft alignment marker 114 and the direction of view dial indicator 108 to the minus on the direction of view scale 107, introduce the variable direction of view endoscope 10 straight into the left nostril of the patient until the endoscope is positioned in the back of the nasal cavity. Once the tip is positioned in the back of the nasal cavity, the direction of view dial is positioned to minus 90 degrees to view the behind the uncinate and into the left maxillary sinus ostia. The left maxillary sinus ostia may be visible with or without minor adjustment of the direction of view or shaft orientation of the endoscope.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that devices and methods within the scope of these claims and their equivalents be covered thereby.

What is claimed is:

1. An endoscopic method for viewing a right maxillary sinus ostia, said method comprising:
adjusting a variable direction of view endoscope to a first direction of view of between about 0 degrees and 20 degrees relative to a longitudinal axis of the endoscope and a shaft orientation to between about 60 degrees and 120 degrees relative to the longitudinal axis of the endoscope;
introducing the variable direction of view endoscope straight into a right nasal cavity of a patient until the endoscope is positioned in the back of the right nasal cavity; and
adjusting the direction of view to a second direction of view of about 60 degrees to 100 degrees relative to the longitudinal axis to view the right maxillary ostia within the right nasal cavity;
wherein the right maxillary ostia may be visible behind an uncinate with or without minor adjustment of the direction of view or shaft orientation of the endoscope.

2. The method of claim 1 wherein the first direction of view is adjusted to between about 5 and 15 degrees relative to the longitudinal axis of the endoscope.

3. The method of claim 1 wherein the second direction of view is adjusted to between about 80 and 90 degrees relative to the longitudinal axis of the endoscope.

4. The method of claim 1 wherein the shaft orientation is adjusted to between about 80 and 100 degrees relative to the longitudinal axis of the endoscope.

5. An endoscopic method for viewing a left maxillary sinus ostia, said method comprising:
adjusting a variable direction of view endoscope to a first direction of view of between about 0 degrees and 20 degrees relative to a longitudinal axis of the endoscope and a shaft orientation to between about −60 degrees and −120 degrees relative to the longitudinal axis of the endoscope;
introducing the variable direction of view endoscope straight into a left nasal cavity of a patient until the endoscope is positioned in the back of the left nasal cavity; and
adjusting the direction of view to a second direction of view of about 60 degrees to 100 degrees relative to the longitudinal axis to view the left maxillary ostia within the left nasal cavity;
wherein the left maxillary ostia may be visible behind an uncinate with or without minor adjustment of the direction of view or shaft orientation of the endoscope.

6. The method of claim 5 wherein the first direction of view is adjusted to between about 5 and 15 degrees relative to the longitudinal axis of the endoscope.

7. The method of claim 5 wherein the second direction of view is adjusted to between about 80 and 90 degrees relative to the longitudinal axis of the endoscope.

8. The method of claim 5 wherein the shaft orientation is adjusted to between about −80 and −100 degrees relative to the longitudinal axis of the endoscope.

9. An endoscopic method for viewing a sphenoid sinus ostia, said method comprising:
adjusting a variable direction of view endoscope to a direction of view of between about 0 degrees and 20 degrees relative to a longitudinal axis of the endoscope; and
introducing the variable direction of view endoscope straight into a nasal cavity of a patient until the endoscope is positioned in the back of the nasal cavity;
wherein the sphenoid sinus ostia may be visible with or without minor adjustment of the direction of view or shaft orientation of the endoscope

10. The method of claim 9 wherein the direction of view is adjusted to between about 5 and 15 degrees relative to the longitudinal axis of the endoscope.

11. An endoscopic method for viewing a sphenoid sinus ostia, said method comprising:
adjusting a variable direction of view endoscope to a direction of view of between about 20 degrees and 70 degrees relative to a longitudinal axis of the endoscope; and
introducing the variable direction of view endoscope straight into a nasal cavity of a patient until the endoscope is positioned below a middle turbinate of the nasal cavity;
wherein the sphenoid sinus ostia may be visible with or without minor adjustment of the direction of view or shaft orientation of the endoscope.

12. The method of claim 11 wherein the direction of view is adjusted to between about 30 and 40 degrees relative to the longitudinal axis of the endoscope.

13. An endoscopic method for viewing a right frontal sinus ostia, said method comprising:
adjusting a variable direction of view endoscope to a first direction of view of between about 0 degrees and 20 degrees relative to a longitudinal axis of the endoscope and a shaft orientation to between about −20 degrees and 20 degrees relative to the longitudinal axis of the endoscope;
introducing the variable direction of view endoscope straight into a right nasal cavity of a patient until the endoscope is positioned in the back of the right nasal cavity; and
adjusting the direction of view to a second direction of view of about 30 degrees to 90 degrees relative to the longitudinal axis to view the right fronal sinus ostia within the right nasal cavity;
wherein the right frontal sinus ostia may be visible with or without minor adjustment of the direction of view or shaft orientation of the endoscope.

14. The method of claim 13 wherein the first direction of view is adjusted to between about 5 and 15 degrees relative to the longitudinal axis of the endoscope.

15. The method of claim 13 wherein the second direction of view is adjusted to between about 45 and 90 degrees relative to the longitudinal axis of the endoscope.

16. The method of claim 13 wherein the shaft orientation is adjusted to between about −10 and 10 degrees relative to the longitudinal axis of the endoscope.

17. An endoscopic method for viewing a left frontal sinus ostia, said method comprising:
adjusting a variable direction of view endoscope to a first direction of view of between about 0 degrees and 20 degrees relative to a longitudinal axis of the endoscope and a shaft orientation to between about −20 degrees and 20 degrees relative to the longitudinal axis of the endoscope;
introducing the variable direction of view endoscope straight into a left nasal cavity of a patient until the endoscope is positioned in the back of the left nasal cavity; and
adjusting the direction of view to a second direction of view of about 30 degrees to 90 degrees relative to the longitudinal axis to view the left fronal sinus ostia within the left nasal cavity;
wherein the left frontal sinus ostia may be visible with or without minor adjustment of the direction of view or shaft orientation of the endoscope.

18. The method of claim 17 wherein the first direction of view is adjusted to between about 5 and 15 degrees relative to the longitudinal axis of the endoscope.

19. The method of claim 17 wherein the second direction of view is adjusted to between about 45 and 90 degrees relative to the longitudinal axis of the endoscope.

20. The method of claim 17 wherein the shaft orientation is adjusted to between about −10 and 10 degrees relative to the longitudinal axis of the endoscope.

21. An endoscopic method for viewing a target portion of a patient's anatomy with access through a nasal cavity, said method comprising:
adjusting a variable direction of view endoscope to a direction of view of between about 0 degrees and 20 degrees relative to a longitudinal axis of the endoscope and a shaft orientation pointing toward the target portion of the patient's anatomy;
introducing the variable direction of view endoscope straight into the nasal cavity of a patient until the endoscope is positioned in the back of the nasal cavity; and
adjusting the direction of view and optionally the shaft orientation to view the target portion of the patient's anatomy.

22. An endoscopic method for viewing a target portion of a patient's anatomy with access through the oral cavity, said method comprising:
adjusting a variable direction of view endoscope to a direction of view of between about 0 degrees and 20 degrees relative to a longitudinal axis of the endoscope and a shaft orientation pointing toward the target portion of the patient's anatomy;
introducing the variable direction of view endoscope straight into the oral cavity of a patient until the endoscope is positioned in the back of the oral cavity; and
adjusting the direction of view and optionally the shaft orientation to view the target portion of the patient's anatomy.
